(19) United States
(12) Patent Application Publication
(10) Pub. No.: US 2006/0217571 A1
(43) Pub. Date: Sep. 28, 2006

(54) Method for producing formic acid formiates

(75) Inventors: Alexander Hauk, Ludwigshafen (DE); Christoph Adami, Weinheim (DE); Jorn Karl, Ludwigshafen (DE); Robert Lenz, Neustadt/Weinstrasse (DE)

Correspondence Address: CONNOLLY BOVE LODGE & HUTZ LLP, SUITE 800, 1990 M STREET NW, WASHINGTON, DC, 20036-3425, US

(73) Assignee: BASF Aktiengesellschaft, Ludwigshafen (DE)

(21) Appl. No.: 10/556,393

(22) PCT Filed: May 14, 2004
(86) PCT No.: PCT/EP04/05214

§ 371 Date: Nov. 10, 2005

(30) Foreign Application Priority Data
May 14, 2003 (DE) 103 21 733.9

(51) Int. Cl. C07C 51/15

(52) U.S. Cl. 562/551

(57) ABSTRACT

A process is proposed for preparing acid formates by providing a liquid stream I containing formic acid and a liquid stream II containing a metal formate, and mixing the liquid stream I with the liquid stream II, obtaining the corresponding acid formate, which comprises feeding, to the liquid stream I or a precursor thereof, to the liquid stream II or a precursor thereof, or to the mixture of the liquid streams I and II, a liquid stream III containing the following components at contents of in each case >0.1% by weight: metal formate, metal methoxide, methanol and MeFo (MeFo), which is produced in a process for preparing MeFo by carbonylating methanol in a reactor at a pressure up to 50 MPa absolute in the presence of a metal methoxide as homogeneous catalyst.

The invention relates to a process for preparing acid formates and to the use of the acid formates prepared by this method for preserving and/or acidifying materials of vegetable and/or animal origin, for treating biowastes and also as additive in animal nutrition, or as growth promoter for animals.

Acid formates have an antimicrobial activity and are used, for example, for preserving and acidifying materials of vegetable and animal origin, for instance grasses, agricultural products or meat, for treating biowastes, or as additive for animal nutrition.

Acid formates and preparation methods for these have long been known. Thus, Gmelins Handbuch der anorganischen Chemie [Gmelin's handbook of inorganic chemistry], 8th Edition, Number 21, pages 816 to 819, Verlag Chemie GmbH, Berlin 1928, and Number 22, pages 919 to 921, Verlag Chemie GmbH, Berlin 1937, describes the synthesis of sodium diformate or of potassium diformate by dissolving sodium formate or potassium formate in formic acid. By lowering the temperature, or by evaporating off excess formic acid, the crystalline diformates can be obtained.

DE 424 017 describes the preparation of acid sodium formates of varying acid content by introducing sodium formate into aqueous formic acid in a corresponding molar ratio.

DE-A 198 43 697.1 discloses a process which makes it possible to prepare acid formates on an industrial scale in a high space-time yield, with great flexibility with respect to composition, and using readily available raw materials and permits a simple processing arrangement with low capital costs and low energy requirements. The process starts from methyl formate termed MeFo hereinafter for short. By means of partial hydrolysis of MeFo, a formic-acid-containing stream is obtained and the non-hydrolyzed portion of MeFo is converted to the corresponding metal formate by saponification. By mixing the formic acid and the metal-formate-containing stream, the acid formate is obtained. Alternatively, it is also possible, in accordance with the process of DE-A 102 37 380.9, to provide the metal-formate-containing stream by a different method to saponifying MeFo, in particular by carbonylating a metal hydroxide in the presence of methanol.

MeFo is an important intermediate in the production of formic acid and is produced industrially by continuous carbonylation of methanol in the liquid phase in the presence of sodium methoxide or potassium methoxide as catalyst at temperatures in the range from about 50 to 150° C. (see Ullmann's Encyclopedia of Industrial Chemistry, 6th Edition, 2000 electronic release, Chapter “FORMIC ACID—Production”). The reaction is a homogeneously catalyzed equilibrium reaction in which, with increasing carbon monoxide partial pressure and decreasing temperature, the equilibrium is shifted in the direction of MeFo. The known processes are operated at a pressure of up to 30 MPa absolute and a temperature of from 50 to 150° C.

In said production of MeFo, in particular, two unwanted side reactions occur which can lead to serious problems in the continuously operated process. Both side reactions lead to the formation of alkali metal formate. Thus, the alkali metal methoxide used reacts with any traces of water introduced in a hydrolysis reaction to form of alkali metal formate and methanol. Furthermore, the alkali metal methoxide used also reacts with MeFo present, forming alkali metal formate and dimethyl ether. The alkali metal formate can then, owing to its inadequate solubility in the reaction medium, lead to deposits in the apparatuses and pipelines up to blockage of pipes and valves. The risk of salt precipitation is particularly high at a high methanol conversion rate, and thus at a high MeFo concentration and can therefore be decreased, in principle, by establishing a partial conversion, ensuring a low concentration of MeFo. However, this is opposed by the effort to achieve as high as possible a space-time yield and thus rather to establish a high concentration of MeFo.

In the literature, a multiplicity of processes and process variants are discussed which differ owing to different measures to decrease the tendency toward formation of deposits.

For instance, DE patent 926 785 describes a high-pressure process operating at 30 MPa, in which, to decrease the salt precipitation, only a low catalyst concentration of 0.25% by weight of sodium (equivalent to 0.59% by weight of sodium methoxide) is used. In addition, the reactor contents are continuously stirred to keep in suspension the precipitated amounts of salt. The liquid reactor effluent which contains about 90% MeFo is expanded and worked up by distillation.

DE patent 926 785 describes a high-pressure process operating at 30 MPa in which, to reduce the salt precipitation a catalyst low concentration of only 0.25% by weight of sodium (equivalent to 0.59% by weight of sodium methoxide) is used. In addition, the reactor contents are stirred continuously to keep the amounts of salt which have precipitated out in suspension. The liquid reactor effluent which contains about 90% MeFo is expanded and worked up by distillation.

DE-B 1 046 602 describes a continuous two-stage process in the presence of from 0.5 to 5% by weight of alkali metal methoxide at a pressure of from 5 to 30 MPa. Ensuring turbulent flow in the reactor is said to prevent deposition. The total methanol conversion is about 90%. The liquid reactor effluent is expanded and worked up by distillation.

DE-B 1 147 214 describes a high-pressure process in which the reaction takes place in the presence of from 0.12 to 0.3 mol % of alkali metal methoxide in at least two sequential reaction zones distinguished by decreasing temperatures and the carbon monoxide is fed to the reactor in at least two substreams at different heights. Short-term repeated changes in the carbon monoxide ratios are to prevent accumulation of salty deposits. The overall methanol conversion rate is up to 97%. The liquid reactor effluent is expanded and worked up by distillation.

WO 96/26178 describes a high-pressure process in which the reaction takes place in the presence of 0.05 to 0.2% by weight of alkali metal methoxide at a pressure of from 21 to 25 MPa. By means of good dispersion of the carbon monoxide, for example by a jet nozzle, despite the low catalyst concentration, a sufficiently high conversion rate can be achieved. For instance, the MeFo concentration in the reactor effluent is up to 97% by weight. The liquid reactor effluent is expanded and worked up by distillation.

DE-A 2 243 811 describes a process in which the reaction takes place in the presence of from 0.4 to 1.5% by weight of alkali metal methoxide in a countercurrent procedure at a pressure of from 4 to 30 MPa and which has a plurality of series-connected reaction zones. Preferred apparatuses are, in particular, columns having flooded trays. The reaction mixture produced in the column bottom contains from 20 to 70% by weight of MeFo and is worked up by distillation after the expansion.

EP-A 0 617 003 describes a process in which the reaction takes place in the presence of from 0.4 to 1.5% by weight of alkali metal methoxide at a pressure of from 1 to 30 MPa. The reactants are first combined in a mixing zone and reacted at least in part. The resultant reaction solution is finally saturated with carbon monoxide and fed to a post-reaction zone without feed of further starting materials. The liquid reactor effluent is expanded and worked up by distillation.

WO 01/07392 describes a process in which the reaction takes place in the presence of from 0.05 to 0.5% by weight of alkali metal methoxide at a carbon methoxide pressure of from 9 to 18 MPa. The liquid reactor effluent which contains from about 60 to 95% by weight of MeFo is fed to a distillation column to separate off the MeFo. The remaining catalyst- and methanol-containing bottom stream is recirculated, residual catalyst and catalyst breakdown products being taken off from a substream thereof via a desalting apparatus. In the exemplary embodiments, space-time yields in the range of from 370 to 880 g/l·h of MeFo were achieved.

U.S. Pat. No. 4,661,624 describes a two-stage process with recirculation of the catalyst-containing methanolic solution. The reaction takes place at a pressure of from 0.48 to 6.9 MPa (from 70 to 1000 psia) and at a concentration of alkali metal methoxide of from 1 to 8 mol % (equivalent to from 1.7 to 13.5% by weight of sodium methoxide). In the second stage, further methanol is fed in countercurrent to react with the residual carbon monoxide. The process is operated at an extremely low conversion rate, so that the liquid reactor effluent contains only from about 2 to 20 mol % of MeFo. The effluent is fed to a distillation column to remove the MeFo. The methanol concentration which is thus very high in the reaction mixture counteracts precipitation of salt-like deposits. However, disadvantages of this process are the associated high energy consumption for removing the MeFo by distillation and the handling of large streams as a result of the high dilution, so that overall an unfavorable energy balance results for the overall process. In addition, to maintain the necessary high catalyst concentration, large amounts of fresh catalyst must continuously be fed.

U.S. Pat. No. 4,216,339 discloses a process in which the reaction takes place in the presence of from 0.2 to 4% by weight of alkali metal methoxide at a pressure of from 2 to 11 MPa and in which the carbon monoxide which is fed is dispersed into the liquid reaction mixture via a self-priming jet nozzle which is operated at a sufficiently high circulation flow rate. In accordance with the amount of starting materials fed, a corresponding amount of reaction mixture is continuously taken off from the liquid circulation stream and worked up by distillation after the expansion. In Example 2, at a pressure of 4.4 MPa in the reaction mixture, an MeFo concentration of 51% by weight was obtained. Even in this process, the high energy consumption for removing the MeFo from the reaction mixture by distillation is disadvantageous because of high dilution. In addition, a large stream of the reaction mixture must be continuously circulated by pumping. Therefore, overall, there is an unfavorable energy balance for the overall process.

DE patent 863 046 describes a continuous low-pressure process in which methanol and from 1 to 2% by weight of sodium (equivalent to from 2.3 to 4.7% by weight of sodium methoxide) are fed from top to bottom in a bubble column equipped with random packings and carbon monoxide is fed from bottom to top in countercurrent and are reacted at a pressure of from about 2.5 to 3.0 MPa (from 25 to 30 atmospheres). The reaction mixture is continuously taken off at the bottom of the reactor and passed for workup by distillation. The gas taken off at the top of the reactor is passed through a cooler, freed from entrained MeFo in a separator and, mixed with fresh carbon monoxide, recirculated to the reactor to ensure sufficiently high gas-introduction stream. The MeFo which is condensed out of the gas phase in the separator is likewise fed to the workup by distillation. Pressure and temperature are to be set in such a manner that the catalyst and its breakdown products are kept in solution.

A process which has been improved in comparison with DE 863 046 is described in DE-B 880 588. In this improved low-pressure process, methanol and from 1.6 to 2.5% by weight of sodium (equivalent to from 3.8 to 5.9% by weight of sodium methoxide) are fed from bottom to top in a bubble column fitted with random packings cocurrently with carbon monoxide and are reacted at a pressure of up to 3.0 MPa (up to 30 atmospheres). Liquid reaction mixture is taken off from a gas dome situated at the reactor top and fed back to the reactor bottom via a circulation pump. The gaseous phase is taken off at the top end of the gas dome, passed through a cooler, then freed from entrained MeFo in a separator and recirculated to the reactor, mixed with fresh carbon monoxide, to ensure a sufficiently high gas-introduction stream. In contrast to the process described in DE 863 046, all of the MeFo is taken off via the gas phase and fed for workup by distillation after the condensation.

The example given in DE 880 588 implies that, using the 770 L reactor (length 8 m and clear width 35 cm) at 3.0 MPa and from 85 to 88° C., in long-term operation, 3.1 kg of MeFo were obtained per hour. This is equivalent to a space-time yield of only 4 g/l·h of MeFo. The MeFo concentration in the condensed crude effluent was about 60% by weight (from 38 to 40% by weight of methanol). The recirculated gas rate of 206 cm³/s, calculated under reaction conditions (equivalent to 17 m³ at 0° C. and 0.1 MPa), and the reactor cross section of 962 cm² give a superficial gas velocity in the pipe of about 0.21 cm/s.

DE-A 102 17 528.4 describes a process for preparing MeFo by reacting methanol with carbon monoxide at a pressure of from 0.5 to 10 MPa absolute and a temperature of from 50 to 150° C. in the presence of a metal alkoxide as catalyst in a reactor in which a gas stream is taken off from the reactor, entrained MeFo is removed from the gas stream by condensation and the remaining gas stream is completely or partially recirculated to the reactor as circulated gas stream, at least in a region of the reactor in which the gas essentially flows in one direction, a mean superficial gas velocity of from 1 to 20 cm/s being set. This process is particularly advantageous with respect to capital costs and energy costs, the catalyst consumption and also space-time yield of MeFo, which is 100 g/l·h.

In all processes described above for preparing MeFo, as unwanted byproduct, the corresponding metal formate forms by hydrolysis of the catalyst made up and by reaction of same with the product of value MeFo. The disadvantages of the metal formate formation, in particular the formation of deposits in the apparatuses and the loss product of value have already been discussed at the outset.

It is an object of the present invention, therefore, to utilize the metal formate which is an unwanted byproduct in the process for preparing MeFo by coupling in a process for preparing acid formates. By means of the inventive utilization of a coupling stream from the MeFo process in the synthesis of acid formates, in particular disposal costs in the MeFo process are reduced, and also material costs in the process for preparing acid formates are reduced.

We have found that this object is achieved in a process for preparing acid formates, in which
a liquid stream I containing formic acid and
a liquid stream II containing a metal formate are provided and
   the liquid stream I is mixed with the liquid stream II, obtaining the corresponding acid formate, which comprises feeding, to the liquid stream I or a precursor thereof, to the liquid stream II or a precursor thereof, or to the mixture of the liquid streams I and II, a liquid stream III containing the components below at contents of in each case >0.1% by weight:
metal formate,
metal methoxide,
methanol and
methyl formate (MeFo),
   which is produced in a process for preparing MeFo by reacting methanol with carbon monoxide, which is compressed in a dry-running compressor, in a reactor in the presence of a metal methoxide as homogeneous catalyst with
withdrawal of the reaction mixture as gas stream in the upper region of the reactor and
withdrawal of a liquid side stream from the reactor as stream III, or with
withdrawal of the reaction mixture as liquid stream IV, by
separating off MeFo and methanol from stream IV.

It has thus been found that a coupling stream from the process for preparing MeFo under the above-defined conditions can be utilized, that is to say:

The carbonylation reaction of the methanol must be carried out using a dry-running compressor for compressing the carbon monoxide to rule out oil fouling due to use of an oil-lubricated compressor.

The catalyst used for the carbonylation must be a metal methoxide to prevent materials foreign to the process from being introduced via the coupling stream into the process for preparing acid formates.

Under the above prerequisits, the coupling stream from the process for preparing MeFo, termed liquid stream III above, exclusively contains materials which are already present in the process for preparing acid formates.

The use of dry-running compressors for compressing carbon monoxide is possible in general in processes for preparing MeFo which operate at pressures up to about 50 bar absolute.

Furthermore, the coupling stream to be used is not the entire stream of the reaction mixture from the MeFo preparation, but a stream which, in comparison, is concentrated with respect to the content of metal formate.

In processes for preparing MeFo with withdrawal of the reaction mixture of the gas stream in the upper region of the reactor, such a coupling stream arises as liquid side stream from the reactor.

In processes for preparing MeFo with withdrawal of the reaction mixture as liquid stream IV, a suitable coupling stream (liquid stream III) is obtained by separating off MeFo and methanol from stream IV.

Said separation of MeFo and methanol from the liquid stream IV can preferably be carried out by distillation in a column by direct introduction of steam.

Advantageously, the acid formate prepared is acid potassium formate, acid sodium formate, acid calcium formate or mixtures thereof, potassium diformate, sodium diformate, sodium tetraformate or mixtures thereof.

Preferably, the liquid stream III is produced as a side stream from a reactor in which MeFo is prepared carbonylating methanol with carbon monoxide at a pressure of from 0.5 to 10 MPa absolute and a temperature of from 50 to 150° C., in which a gas stream is withdrawn from the reactor from the upper region of same, entrained MeFo is separated off from the gas stream by condensation, and the remaining gas stream is completely or partially recirculated as circulated gas stream to the reactor, a mean superficial gas velocity of at least from 1 to 20 cm/s, preferably from at least 3 to 10 cm/s, being set in at least one region of the reactor in which the gas flows in essentially one direction.

A process of this type is described in DE-A 102 17 528.4, the disclosure of which is expressly hereby fully incorporated in the present application by reference.

Preferably, the metal methoxide is used as homogeneous catalyst in the carbonylation of methanol by carbon monoxide in a concentration of from 0.01 to 2 mol/kg of liquid reaction mixture, the metal methoxide selected preferably being potassium methoxide.

Methanol is preferably carbonylated to form MeFo at a pressure of from 2 to 4 MPa absolute and at a temperature of from 60 to 85° C.

A preferred molar ratio of the total amount of the methanol fed to the reactor and the amount of carbon monoxide freshly fed is set in the range from 1.4 to 3.3 to 1, preferably to 2 to 1.

Preferably, the reactor used for preparing MeFo is a bubble column and this is operated with respect to feeding the methanol-containing liquid stream and the carbon monoxide-containing gas stream in the cocurrent procedure.

Alternatively, it is possible to use, as reactor for preparing MeFo, a cascade reactor and preferably to operate the uppermost zone of the cascade reactor at a temperature of from 80 to 150° C.

In an advantageous process variant, the gas stream withdrawn from the reactor can, in an enrichment column, be divided into an MeFo-containing bottom stream and a carbon monoxide-containing and MeFo-containing top stream, from which top stream entrained MeFo is removed by condensation and the remaining gas stream is recirculated completely or partially to the reactor as circulated gas stream.

The processes for preparing acid formates in which the liquid stream III from the preparation of MeFo is utilized are in principle not limited. In general, processes suitable therefor are all of those by which a liquid stream I containing formic acid and a liquid stream II containing a metal formate are prepared and the liquid stream I is mixed with the liquid stream II, obtaining the corresponding acid formate.

Preference is given to processes for preparing acid formates by which the liquid stream I is obtained starting from MeFo in which
(a) MeFo is partially hydrolyzed and
(b) the reaction mixture of the partial hydrolysis from process stage (a) is separated by distillation into a lower-boiling stream V, comprising MeFo and methanol, and the higher-boiling stream I, comprising formic acid and water.

Processes of this type are disclosed by German patent applications DE-A 102 37 379.5 and DE-A 102 37 380.9, the disclosure of which is hereby incorporated in full in the present application by reference.

Acid formates are compounds and mixtures which contain formate anions (HCOO⁻), cations (M^{x+}) and formic acid (HCOOH). They can be present together in the form of a solid or a liquid and may also comprise other components, for example other salts, additives or solvents, such as water. Generally, the acid formates are represented by the general formula
HCOO⁻M^{X+}_{1/x}*y HCOOH (I),
where M is a monovalent or polyvalent inorganic or organic cation, x is a positive integer and indicates the charge of the cation and y represents the molar fraction of formic acid based on the formate anion. The molar fraction of formic acid based on the formate anion y is generally from 0.01 to 100, preferably from 0.05 to 20, particularly preferably 0.5 to 5, and in particular from 0.9 to 3.1.

The nature of the inorganic or organic cation M^{x+} is in principle unimportant, provided that it is stable under the conditions under which the acid formate is handled. This also includes, for example, stability with respect to the formate ion which has a reducing action. Possible inorganic cations are the monovalent and/or polyvalent metal cations of metals from groups 1 to 14 of the Periodic Table of the Elements, for example lithium (Li⁺), sodium (Na⁺), potassium (K⁺), cesium (Cs⁺), magnesium (Mg²⁺), calcium (Ca²⁺), strontium (Sr²⁺) and barium (Ba²+), preferably sodium (Na⁺), potassium (K⁺), cesium (Cs⁺) and calcium (Ca²⁺). Possible organic cations are unsubstituted ammonium (NH₄⁺) and ammonium substituted by one or more carbon-containing radicals which may also be bound to one another, for example methylammonium, dimethylammonium, trimethylammonium, ethylammonium, diethylammonium, triethylammonium, pyrrolidinium, N-methylpyrrolidinium, piperidinium, N-methylpiperidinium or pyridinium.

A carbon-containing organic radical means an unsubstituted or substituted aliphatic, aromatic or araliphatic radical having from 1 to 30 carbons. This radical can contain one or more heteroatoms, such as oxygen, nitrogen, sulfur or phosphorus, for example —O—, —S—, —NR—, —CO—, —N═, —PR— and/or —PR₂ and/or be substituted by one or more functional groups which contain, for example, oxygen, nitrogen, sulfur and/or halogen, for example by fluorine, chlorine, bromine, iodine and/or a cyano group (the radical R is likewise in this case a carbon-containing organic radical). The carbon-containing organic radical can be a monovalent or polyvalent, for example divalent or trivalent, radical.

The individual processes stages are described in more detail below:

Preparation of the Liquid Stream I

Process Stage (a)

In the process stage (a), MeFo is partially hydrolyzed with water to formic acid and methanol. Partial means that only a portion of the MeFo supplied is hydrolyzed.

In process stage (a), the processes known per se for hydrolyzing MeFo can be used. A general overview of known and industrially relevant processes for hydrolysis is given, for example, in Ullmann's Encyclopedia of Industrial Chemistry, 6th Edition, 2000 electronic release, Chapter “FORMIC ACID, Production”. Further suitable hydrolysis processes are also described, for example, in EP-A 0 005 998 and EP-A 0 017 866.

The hydrolysis is generally carried out at a temperature from 80 to 150° C. and a pressure from 0.5 to 2.0 MPa absolute. The reaction apparatuses which can be used are in principle all reaction apparatuses which are suitable for reactions in the liquid phase. Examples are stirred tanks and jet loop reactors. Preference is given to the use of a cascade reactor.

Generally it is advantageous to carry out the hydrolysis in the presence of an acid catalyst, since this significantly increases the hydrolysis rate. Acid catalysts which can be used are the formic acid formed or additional catalysts. The additional catalysts can be of homogeneous or heterogeneous nature. Examples of heterogeneous catalysts are acid ion exchangers, such as polysulfonic acids or poly(perfluoroalkylene)sulfonic acids (for example Nafion® from Du Pont) and examples of homogeneous catalysts are strong inorganic or organic acids, such as sulfuric acid, hydrochloric acid or alkylsulfonic and tolylsulfonic acids. If homogeneous catalysts are used, these must generally be separated off in a following stage. Depending on the desired purity of the acid formates to be prepared, however, it is also possible to leave them in the system. In this case, the acid catalysts are usually encountered in the form of their salts in the acid formate. Particularly preferably, the partial hydrolysis is carried out in the presence of formic acid as acid catalyst, which avoids the addition of an additional catalyst and its subsequent removal or possible contamination of the acid formates. Generally, at the reactor inlet, for this purpose, a formic acid concentration of from about 0.1 to 2% by weight, based on the liquid water and MeFo-containing mixture present, is set by targeted addition of formic acid, or of a formic-acid-containing stream.

The molar ratio of water to MeFo to be used in the hydrolysis is advantageously generally from 0.1:1 to 10:1, preferably from 6:1 to 3:1. Since an equilibrium reaction is involved, preferably an excess of water is added, as also implied, for example, by EP-A 0 017 866.

The reaction mixture obtained from the partial hydrolysis thus contains unreacted MeFo, formic acid, methanol and also, owing to the preferred use of a water excess, water. Preferably, the aqueous reaction mixture contains from 5 to 15 mol %, particularly preferably from 8 to 12 mol %, of formic acid, from 3 to 30 mol %, particularly preferably from 6 to 12 mol %, of MeFo and from 6 to 15 mol %, particularly preferably from 8 to 12 mol %, of methanol.

Process Stage (b)

In the process stage (b), MeFo and methanol (stream V) are separated off from the reaction mixture obtained in process stage (a) by distillation, forming the stream containing formic acid and water. MeFo and methanol can in principle be separated off together in the form of a stream or separately in the form of an MeFo-containing stream and a methanol-containing stream. Generally, MeFo and methanol are take off separately or together in the upper part of the column. The stream I containing formic acid and water is generally taken off from the bottom. Preference is given in process stage (b) to the joint removal of a MeFo- and methanol-containing stream.

The design and operation of the distillation column are primarily dependent on the composition of the stream fed and on the desired purities of the two product streams and can be determined by those skilled in the art in a known manner. Preferably, the MeFo- and methanol-containing relatively low-boiling liquid stream V can be recirculated to the process stage (a).

Preparation of the Liquid Stream II

In one process variant, the liquid stream II containing a metal formate can be obtained by saponification, by the MeFo- and methanol-comprising stream V, in process stage (c), being
(i) reacted with a basic compound having a pKₐ of the conjugate acid of the corresponding dissociation state of ≧3, measured at 25° C. in aqueous solution, in the presence of water, and
(ii) converting it into the metal-formate- and water-containing stream II by separating off the methanol by distillation.

The basic compound to be used preferably has a pKₐ of the conjugate acid of the corresponding dissociation state of ≧3.5, particularly preferably ≧9, and very particularly preferably ≧10, measured at 25° C. in aqueous solution. The basic compound can be of inorganic or organic nature. The basic compound can be a salt or a covalent compound. The conjugate acid of the corresponding dissociation state here is an acid formed by formal addition of a proton (H⁺).

In the event that the basic compound is a salt, this can in general be represented by the formula
M^{x+}ₐA^{a-}ₓ (II),
where M and x have the meaning specified under (I) and A is an inorganic or organic anion having the charge “a−”. The conjugate acid of the corresponding dissociation state thus corresponds to HA^{(a-1)-}. The corresponding dissociation equation, which defines the pKₐ to be used, is:

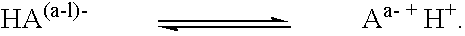

In the event that the basic compound is a covalent compound B, the dissociation equation which defines the pKₐ to be used is:

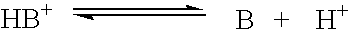

Examples of suitable basic compounds are the salts M^{x+}ₐA^{a-}ₓ (II), where Mₓ₊ is a monovalent or polyvalent metal cation of a metal as described above and A^{a-} is an anion as listed in Table 1a and the covalent compounds B are as listed in Table 1b.

TABLE 1a
Possible anions A^{a-} of suitable basic compounds and
pKₐs (measured at 25° C. in aqueous solution)
of the conjugate acids of the corresponding dissociation states.

| Anions A^{a-} | Conjugate acid | pKₐ |
|---|---|---|
| Hydroxide (OH⁻) | Water (H₂O) | 14.0 |
| Carbonate (CO₃²⁻) | Hydrogen carbonate (HCO₃⁻) | 10.3 |
| Hydrogen carbonate (HCO₃⁻) | Carbonic acid (H₂CO₃) | 6.4 |
| Borate (BO₃⁻) | Hydrogen borate (HBO₃²⁻) | >14 |
| Hydrogen borate (HBO₃²⁻) | Dihydrogen borate (H₂BO₃⁻) | >14 |
| Dihydrogen borate (H₂BO₃⁻) | Boric acid (H₃BO₃) | 9.3 |
| Phosphate (PO₄³⁻) | Hydrogen phosphate (HPO₄²⁻) | 12.3 |
| Hydrogen phosphate (HPO₄²⁻) | Dihydrogen phosphate (H₂PO₄⁻) | 7.2 |
| Formate | Formic acid | 3.8 |
| Acetate | Acetic acid | 4.8 |
| Propionate | Propionic acid | 4.9 |
| Oxalate (C₂O₄²⁻) | Hydrogen oxalate (HC₂O₄⁻) | 4.2 |
| 2-Ethylhexanoate | 2-Ethylhexanoic acid | >4 |
| (C₄H₉—CH(C₂H₅)—COO⁻) | (C₄H₉—CH(C₂H₅)—COOH) | |

TABLE 1b
Possible covalent bases B as suitable basic compounds and
pKₐs (measured at 25° C. in aqueous solution)
of the conjugate acids of the corresponding dissociation
states.

| Covalent base B | Conjugate acid | pKₐ |
|---|---|---|
| Ammonia | Ammonium | 9.3 |
| Methylamine | Methylammonium | 10.6 |
| Dimethylamine | Dimethylammonium | 10.7 |
| Trimethylamine | Trimethylammonium | 9.8 |
| Ethylamine | Ethylammonium | 10.7 |
| Diethylamine | Diethylammonium | 11.0 |
| Triethylamine | Triethylammonium | 10.8 |
| Pyrrolidine | Pyrrolidinium | 11.3 |
| N-Methylpyrrolidine | N-Methylpyrrolidinium | 10.3 |
| Piperidine | Piperidinium | 11.1 |
| N-methylpiperidine | N-methylpiperidinium | 10.1 |
| Pyridine | Pyridinium | 5.3 |

Preferably, the basic compound used is lithium hydroxide, lithium hydrogen carbonate, lithium carbonate, sodium hydroxide, sodium hydrogen carbonate, sodium carbonate, potassium hydroxide, potassium hydrogen carbonate, potassium carbonate, ammonium carbonate, ammonium hydrogen carbonate and/or ammonia, particularly preferably sodium hydroxide, sodium hydrogen carbonate, sodium carbonate, potassium hydroxide, potassium hydrogen carbonate, potassium carbonate, and/or ammonia, and very particularly preferably sodium hydroxide, sodium carbonate, potassium hydroxide and/or potassium carbonate, in particular sodium hydroxide and/or potassium hydroxide.

The manner in which the basic compounds are added is generally unimportant. They can be added in solid, liquid or gaseous form, as pure substance, as mixture of substances or as solution. Examples which may be mentioned are addition in the form of aqueous solutions (for example aqueous solutions of the alkali metal salts or ammonia water), in the form of solid compounds (for example powders of the alkali metal salts), in the gaseous state (for example gaseous ammonia). Preference is given to addition in the form of their aqueous solutions.

The sequence in which the starting materials are added is also in general unimportant in the inventive process. Thus, it is possible, for example, to introduce first the basic compound in solid or liquid form (for example as aqueous solution) and then to introduce the MeFo-containing stream in the liquid or gaseous state with stirring. It is also possible to introduce first the MeFo-containing stream in liquid form and then to add the basic compound. Furthermore, it is of course also possible, and advantageous, in particular when a continuous process is being carried out, to combine the MeFo-containing Stream and the basic compound continuously.

The molar ratio of the MeFo to the basic compound is advantageously to be set stoichiometrically, that is to say in such a manner that the added MeFo reacts with the added basic compound to give the corresponding formate and water in accordance with the reaction stoichiometry. The critical parameter of this is what is termed the molar equivalent of the basic compound, which must take into account in this case all dissociation states which lead by addition or protons to conjugate acids which have a pKₐ of ³3, measured at 25° C. in aqueous solution. Thus, when potassium hydroxide is used as basic compound, preferably an MeFo/potassium hydroxide molar ratio of 1.0 is to be selected, since this corresponds to the formation of potassium formate:

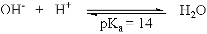

When potassium carbonate is used as a basic compound, preferably an MeFo/potassium carbonate molar 2.0 is to be selected, since the conjugate acid is dibasic:

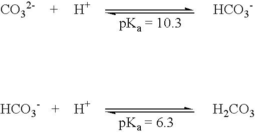

Deviations upward and downward from said stoichiometric addition are, however, also possible in the inventive process. Thus, in the event of a deficiency of basic compound, there is the risk of incomplete reaction of the MeFo and thus the risk of contaminating the methanol, which is to be removed by distillation, with unreacted MeFo. In the event of a excess of basic compound, the resultant stream would, in addition to the corresponding formate and the water, further contain the residual basic compound.

The amount of water to be used in process of stage (c) can vary over a broad range. Generally, in the reaction, from 20 to 90% by weight, and preferably from 40 to 50% by weight, of water is used, based on the amount of MeFo which is fed. Generally, the water is added via an aqueous solution of the basic compound, although the addition of pure water is also possible.

The reaction of the MeFo-containing stream in the process stage (c) with said basic compound in the presence of water is generally carried out at a temperature of from 0 to 150° C., preferably from 30 to 120° C., and particularly preferably from 50 to 80° C. In the procedure, the pressure is generally from 0.05 to 1 MPa absolute, preferably from 0.08 to 0.5 MPa absolute, and particularly preferably from 0.09 to 0.15 MPa absolute.

The reaction of the MeFo-containing stream in process stage (c) with said basic compound in the presence of water is in principle independent of the removal of methanol by distillation. The methanol can therefore be removed by distillation, in principle, before, together with or after said reaction. Preferably the methanol is removed by distillation together with or after said reaction.

In the removal of the methanol by distillation before or after said reaction, reaction apparatuses which can be used for the reaction are in principle all which are suitable for reactions in the liquid phase. Examples are stirred tanks and jet loop reactors. The methanol is removed by distillation then in a separate step, customarily in a distillation column.

In the inventive process, particular preference is given to removing the methanol by distillation (process stage (ii)) together with the reaction of the MeFo with the water and the basic compound, with transfer into the stream II containing formate and water (process stage (i)) in a column.

Owing to the lower boiling point of MeFo compared with water, in this case, the stream containing MeFo and methanol from process stage (b) is advantageously added below the feed point of the water and the basic compound. Since MeFo and the methanol ascend in the column and the water and the basic compound flow downward, the column has a region suitable for said reaction. The methanol ascends upward and can be isolated overhead. Since MeFo is generally prepared by carbonylating methanol, it is particularly advantageous to recirculate the methanol isolated overhead as feedstock for the MeFo preparation, in which case the methanol to be recirculated, in this variant, can also perfectly possibly contain residual amounts of MeFo. Thus, in the overall balance, it is merely necessary to replace the small methanol losses by fresh methanol.

The stream containing the aqueous formate flows downward in the column and is taken off as bottom stream. It may be advantageous here to withdraw a portion of the water as side stream at the bottom end of the column and recirculate it to the hydrolysis. This measure alone can result in a more highly concentrated aqueous solution of the corresponding formate being obtained.

The required residence time in the saponification part of the column can be provided, for example, by suitable internals, such as Thormann trays, or possibly by an external reaction volume. When an external reaction volume is provided, the stream to be saponified is taken off from the column at a suitable point by a side stream takeoff, fed to the external reaction apparatus, and fed back to the column at a suitable point. Both variants are to be considered as primarily equivalent.

The column is designed in a manner customary for those skilled in the art.

In a further alternative, it is possible to produce the metal-formate-containing stream II by carbonylating the corresponding metal hydroxide. Such a process is described in DE-A 102 347 380.9, the disclosure of which is hereby incorporated in full by reference into the present application.

Said carbonylation proves particularly advantageous, in particular since it makes it possible to use readily and easily accessible starting materials and is technically simple to carry out. Thus, for example, according to A. F. Hollemann, N. Wiberg, Lehrbuch der anorganischen Chemie [Textbook of Inorganic Chemistry], Walter de Gruyter Verlag Berlin New York, 1985, 91.-100. edition, page 722, sodium formate may be prepared by introducing carbon monoxide into sodium hydroxide solution at from 150 to 170° C. and at a pressure from 3 to 4 bar, and according to page 947 of the same textbook, potassium formate may be prepared by reaction of carbon monoxide on an aqueous solution of potassium sulfate and caustic lime at 230° C. and 30 bar. According to Ullmann's Encyclopedia of Industrial Chemistry, 6th edition, 2000 electronic release, Chapter “FORMIC ACID, Production, Other Processes”, sodium formate can be produced, for example, by the action of carbon monoxide on aqueous sodium hydroxide solution at 180° C. and from 1.5 to 1.8 MPa, using a reaction tower. The aqueous sodium hydroxide solution trickles from top to bottom here, whereas the carbon monoxide flows from bottom to top in countercurrent flow.

In the next process step to prepare acid formates, the prepared streams I, containing formic acid, and II, containing a metal formate, are mixed.

Here, the sequence of addition of the liquid stream I and the liquid stream II is generally unimportant. In particular, it is possible, and possibly advantageous, to subject the formic-acid-containing liquid stream I and/or the metal-formate-containing liquid stream II, before mixing, to a concentration of formic acid, or of metal formate respectively, in particular by removing a part of the water present by evaporation, preferably by distillation.

The specific process conditions, in particular with respect to temperature and pressure, and also the apparatuses specifically used for mixing the liquid streams I and II are in principle not subject to any restrictions, and can be determined by those skilled in the art in a suitable manner. The above-cited DE-A 102 37 379.5 contains further details in this respect.

Preferably, the liquid stream I and the liquid stream II are mixed in a column, the acid formate and water-containing bottoms liquid are taken off therefrom and acid formate is precipitated out of the bottoms liquid by crystallization, spray granulation, spray drying or melt crystallization, and the precipitated acid formate is isolated.

However, it is alternatively possible to package as such the acid formate obtained as bottoms liquid in the form of an aqueous solution and to store, transport and/or use it for appropriate formulations and applications.

The procedure of the concentration process lies within the scope of customary skilled knowledge and is presented in detail, for example, in DE-A 102 37 379.

The invention also relates to the use of the acid formates prepared utilizing a coupling stream from the MeEo process for preserving and/or acidifying plant and animal materials.

The invention also relates to the use of the acid formates prepared according to the invention for preserving and/or acidifying plant and animal materials. Examples are the use of acid formates for preserving and acidifying grass, agricultural plants, fish and also fish products and meat products, as are described, for example, WO 97/05783, WO 99/12435, WO 00/08929 and WO 01/19207.

Furthermore, the invention relates to the use of the acid formates prepared according to the invention for treating biowastes. The use of acid formates for treating biowastes is described, for example, in WO 97/20911.

The invention further relates to the use of the acid formates prepared according to the invention as additive in animal nutrition and/or as growth promoter for animals, for example for breeding sows, fattening pigs, poultry, calves, cows and fish. Said use is described, for example, in WO 96/35337. Preference is given to the use of the acid formates prepared according to the invention, in particular potassium diformate, as additive in animal nutrition and/or as growth promoter for animals, in particular for breeding sows and fattening pigs.

Very particularly preferred mixtures for the preferred use of the acid potassium formates prepared according to the inventive process as additive in animal nutrition and/or as growth promoter for animals are the following two compositions.

| | Mixture 1 | Mixture 2 |
|---|---|---|
| | (% by weight) | (% by weight |
| potassium diformate | from 20 to 60 | from 60 to 99 |
| sodium diformate/tetraformate | from 20 to 50 | — |
| calcium formate | from 0 to 25 | from 0 to 28 |
| dessicant (silicate or starch) | from 0 to 4 | from 0 to 4 |
| water | from 0 to 5 | from 0 to 5 |

Very particularly preferably, the potassium diformate prepared according to the invention is used as additive in animal nutrition and/or as growth promoter for animals in the form of a product of the composition 98.0±1% by weight of potassium diformate, 1.5±1% by weight of silicate and 0.5±0.3% by weight of water.

The invention will be described in more detail below with reference to a drawing:

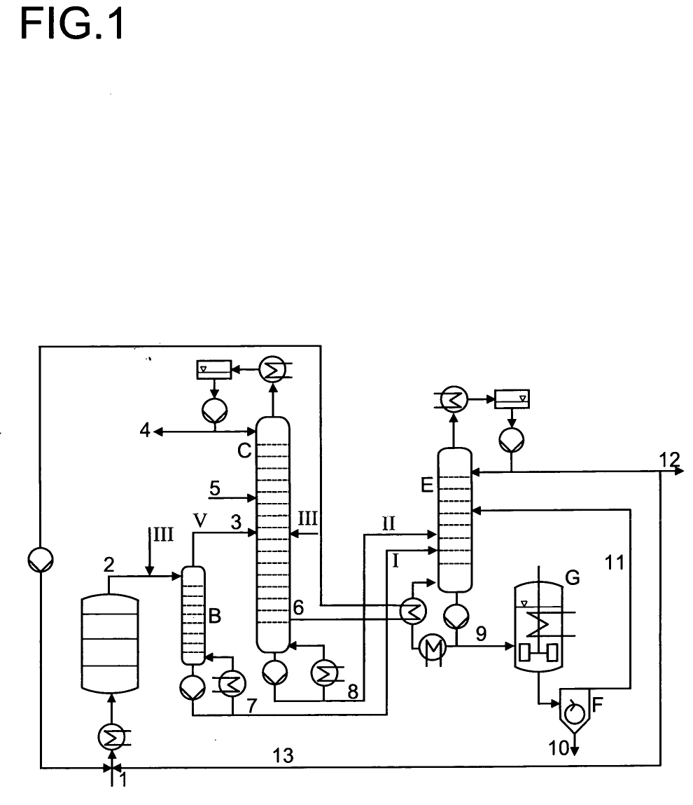

FIG. 1 shows a simplified process of the flow chart of particularly preferred embodiment of the process for synthesizing potassium diformate, with ester saponification and

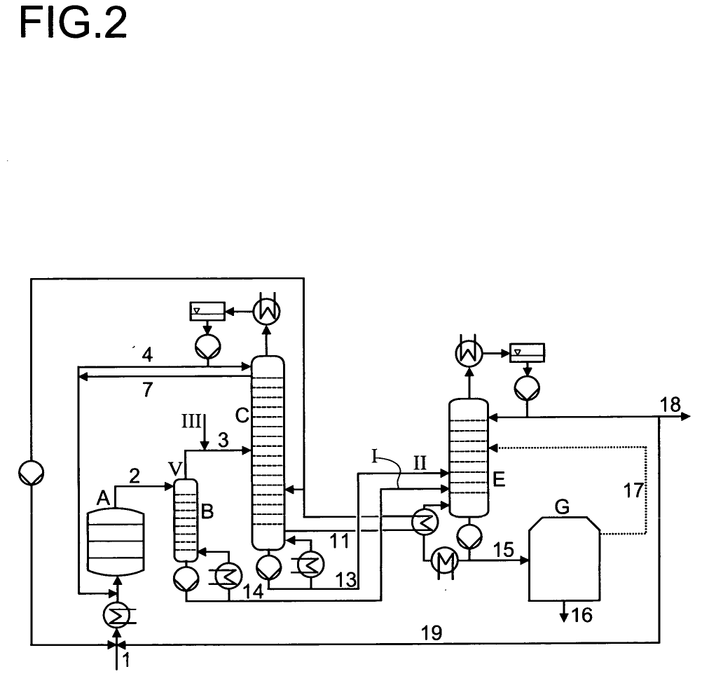

FIG. 2 shows a simplified process of a flow chart of a particularly preferred embodiment in which the metal formate is provided by carbonylation.

In a particularly preferred embodiment, the simplified process flow chart of which is shown in FIG. 1, via line 1, MeFo and formic acid-containing water recirculated from the process are added to the cascade hydrolysis reactor A via line 1. Generally, the two starting materials, premixed (as shown in the flow chart) or separately, are brought to the desired entry temperature in a heat exchanger. The reaction mixture originating from the hydrolysis stage (process stage (a)) which contains unreacted MeFo, water, formic acid and methanol, is fed via line 2 to column B in which the reaction mixture is separated by distillation into a top stream containing MeFo and methanol, and a bottom stream containing aqueous formic acid (process stage (b)). A liquid stream III containing metal formate, metal methoxide, methanol and MeFo is fed to line 2 from a process for preparing MeFo by carbonylation of methanol. The MeFo and methanol-containing top stream V is fed via line 3 to the column C. In addition, the aqueous basic compound, particularly preferably potassium hydroxide solution, is fed to the column C above the feed point of the MeFo- and methanol-containing stream via line 5. Alternatively, or in addition to the feed via line 2, a liquid stream III from the preparation of MeFo is fed to the column C, preferably in about the middle region of same. Methanol is obtained overhead from column C and is preferably recirculated for renewed preparation of MeFo by carbonylation. At the lower end of the column C, a part of the water is taken off and recirculated to the hydrolysis stage via line 6. The bottom product obtained is an aqueous potassium formate solution. The aqueous formic acid-containing stream I from the process stage (b) is fed via line 7 to the column E. The stream II containing the aqueous formate solution from the process stage (c) is fed via line 8. The column E is advantageously operated in such a manner that the bottoms product obtained is a concentrated mixture containing formic acid, formate and water and having a water content of generally from 10 to 40% by weight. A part of the water is taken off from the column E as overhead product in the form a formic acid-containing water stream and recirculated to the hydrolysis stage via line 13. A part of the small amounts of formic acid-containing water stream can here be optionally withdrawn from the system via line 12. The bottoms product of the column E is fed via line 9 to an apparatus G which is suitable for the crystallization, for example what is termed a cooling disk crystallizer. The crystallization is performed primarily by temperature decrease. The resultant crystals are fed together with the supernatant solution to the apparatus F for separation. Preferably, the separation is performed by centrifugation. The crystals separated off are withdrawn via line 10 and can be dried, for example, in optional following stages and/or further processed. The resultant mother liquor is recirculated to the column E via line 11.

In another particularly preferred embodiment, the simplified process flow chart of which is shown in FIG. 2, the process stages (a) and (b), and the preparation of the metal formate, preferably potassium formate, and the operation of the columns C and E are carried out as in the particularly preferred embodiment described above. A liquid stream III from the preparation of MeFo is fed to line 3. Via line 12, a metal formate-containing stream II is fed to the column C, which stream II was obtained by carbonylating the corresponding metal hydroxide. The stream I containing the aqueous formic acid from the process stage (b) is fed via line 14 to the column E and the stream II containing the aqueous metal formate solution from column C is fed via line 13 to the column E. The column E is preferably operated in such a manner that the bottoms product obtained is a concentrated mixture containing formic acid, metal formate and water, having a water content of generally from 0.5 to 30% by weight. A part of the water fed is taken off as overhead product from the column E in the form of a formic acid-containing water stream and recirculated via line 19 to the hydrolysis stage. A part of the water stream containing small amounts of formic acid can here optionally be taken off from the system via line 18. The bottoms product of the column E is fed via line 15 to an apparatus G which is suitable for spray granulation, spray drying or melt crystallization. The resultant solid acid formate is taken off via line 16 and can, for example, in optional following stages, be further dried and/or further processed. The resultant condensate can optionally be recirculated via line 17 to the column E or can be discharged from the system.

We claim:

1. A process for preparing acid formats in which
a liquid stream I containing formic acid and
a liquid stream II containing a metal formate,
are provided and
the liquid stream I is mixed with the liquid stream II, obtaining the corresponding acid formate, which comprises feeding, to the liquid stream I or a precursor thereof, to the liquid stream II or a precursor thereof, or to the mixture of the liquid streams I and II, a liquid stream III containing the components below at contents of in each case >0.1% by weight:
metal formate,
metal methoxide,
methanol and
methyl formate (MeFo),
which is produced in a process for preparing MeFo by carbonylating methanol in a reactor at a pressure up to 50 MPa absolute in the presence of a metal methoxide as homogeneous catalyst, with
withdrawal of the reaction mixture as gas stream in the upper region of the reactor and
withdrawal of a liquid side stream from the reactor as stream III, or withdrawal of the reaction mixture as liquid stream IV, by
separating off MeFo and methanol from stream IV.

2. A process as claimed in claim 1, wherein MeFo are separated off from the liquid stream IV by distillation in a column by direct introduction of steam.

3. A process as claimed in claim 1, wherein the liquid stream I is obtained starting from MeFo, with
(a) MeFo being partially hydrolyzed and
(b) the reaction mixture of the partial hydrolysis from process stage (a) being separated by distillation into a lower-boiling stream V, comprising MeFo and methanol, and the higher-boiling stream I, comprising formic acid and water.

4. A process as claimed in claim 3, wherein water and MeFo are used in the process stage (a) in a molar ratio of from 0.1:1 to 10:1, in particular in a molar ratio of from 6:1 to 3:1.

5. A process as claimed in claim 3, wherein the liquid stream V is recirculated to the process stage (a).

6. A process as claimed in claim 2, wherein the stream V comprising MeFo and methanol is transferred to stream II containing metal formate and water, in process stage (c), by
(i) reaction with a basic compound having a pKₐ of the conjugate acid of the corresponding dissociation stage of ≧3, measured at 25° C. in aqueous solution, in the presence of water, and
(ii) removal of the methanol by distillation.

7. A process as claimed in claim 6, wherein the process stages (i) and (ii) are carried together in one column.

8. A process as claimed in claim 3, wherein the metal formate-containing stream II is produced by carbonylating the corresponding metal hydroxide.

9. A process as claimed in claim 1, wherein the liquid stream I and the liquid stream II are mixed in a column (E), the bottoms liquid containing acid formate and water is taken off, the acid formate is separated off by crystallization, spray granulation, spray drying or melt crystallization, and this acid formate is isolated.

10. A process as claimed in claim 1, wherein the acid formate prepared is acid potassium formate, acid sodium formate, acid calcium formate or mixtures thereof, potassium formate, acid sodium formate, acid calcium formate or mixtures thereof, potassium diformate, sodium diformate, sodium tetraformate or mixtures thereof.

11. A process as claimed in claim 1, wherein the process for preparing MeFo by carbonylating methanol with carbon monoxide in a reactor in which the liquid stream III is produced as a side stream, is carried out at a pressure of from 0.5 to 10 MPa absolute and at a temperature of from 50 to 150° C. in which a gas stream is taken off from the reactor, entrained MeFo is separated off from the gas stream by condensation and the remaining gas stream is completely or partially recirculated to the reactor as circulated gas stream, a mean gas superficial velocity of from at least 1 to 20 cm/s, preferably of from at least 2 to 10 cm/s, being set in at least one region of the reactor in which the gas flows essentially in one direction.

12. A process as claimed in claim 10, wherein the metal methoxide is used as homogeneous catalyst in the carbonylation of methanol with carbon monoxide at a concentration of from 0.01 to 2 mol/kg of liquid reaction mixture, and the metal methoxide used is preferably potassium methoxide.

13. A process as claimed in claim 11, wherein the methanol is carbonylated to form MeFo at a pressure of from 2 to 4 MPa absolute and at a temperature of from 60 to 85° C.

14. A process as claimed in claim 11, wherein a molar ratio of the total amount of the methanol fed to the reactor to the amount of freshly supplied carbon monoxide of from 1.4 to 3.3:1, preferably 2:1, is set.

15. A process as claimed in claim 11, wherein the reactor used for preparing MeFo is a bubble column and this is operated in a cocurrent flow procedure with respect to the feed of the methanol-containing liquid stream and the carbon monoxide-containing gas stream.

16. A process as claimed in claim 11, wherein the reactor used for preparing MeFo is a cascade reactor, and preferably the top zone of the cascade reactor is operated at a temperature of from 80 to 150° C.

17. A process as claimed in claim 11, wherein the gas stream withdrawn from the reactor is separated in an enrichment column into an MeFo-containing bottom stream and a carbon monoxide- and MeFo-containing top stream, entrained MeFo is separated off from the top stream by condensation and the remaining gas stream is completely or partially recirculated to the reactor as circulated gas stream.

18-20. (canceled)

19. A process as claimed in claim 4, wherein water and MeFo are used in the process stage (a) in a molar ratio of from 6:1 to 3:1.

20. A process as claimed in claim 11, wherein a mean gas superficial velocity of from at least 2 to 10 cm/s, is set in at least one region of the reactor in which the gas flows essentially in one direction.

21. A process as claimed in claim 14, wherein a molar ratio of the total amount of the methanol fed to the reactor to the amount of freshly supplied carbon monoxide of 2:1, is set.
